(19) United States
(12) Patent Application Publication
(10) Pub. No.: US 2009/0023204 A2
(43) Pub. Date: Jan. 22, 2009

(54) A Device for the Storage and Treatment of Biodegradable Wet Solid Waste

(75) Inventors: Muley Uddhaorao, Nagpur, MH (IN); Devotta Sukumar, Nagpur, MH (IN)

Correspondence Address: GREENLEE WINNER AND SULLIVAN P C, 4875 PEARL EAST CIRCLE, SUITE 200, BOULDER, CO, 80301, UNITED STATES, 303-499-8080, 303-499-8089, usptomail@greenwin.com

(73) Assignee: Council of Scientific & Industrial Research, New Delhi (IN)

(21) Appl. No.: 11/959,035

(22) Filed: Dec. 18, 2007

(30) Foreign Application Priority Data
Feb. 14, 2007 (IN) 293/Del/2007

(51) Int. Cl. C12M 1/00

(52) U.S. Cl. 435/290.1

(57) ABSTRACT

The present invention relates to a device for the storage and treatment of biodegradable wet solid waste, especially for vegetarian household waste materials. The device consists of a bucket shaped container with a mesh bottom and a removable cover with a hole at the center. The container is provided with a leachate collection basket at the bottom, which is mounted on an iron stand to hold the device. Openings are provided on the lateral surface of the basket for ventilation. The waste is added to the container from the top by hand lifting the lid. The ventilation facilitates degradation of the material in side the container, reducing the release of odour and, leachate. The volume of the waste also gets reduced because of self-compaction. In one embodiment the storage and treatment device is provided in the form of a house hold kit.

### CROSS-REFERENCE TO RELATED APPLICATION

This application claims priority to Indian Patent Application No. 293/Del/2007 filed Feb. 14, 2007, which is incorporated herein by reference to the extent not inconsistent herewith.

### FIELD OF THE INVENTION

The present invention relates to a device for the storage and treatment of biodegradable wet solid waste. In particular, the invention relates to the device for storage and treatment of biodegradable wet solid waste, thus for improving integrated solid waste management system for the biodegradation and recycling of household waste.

### BACKGROUND

The solid waste management system involves collection, transportation, processing, treatment and disposal of waste generated day to day in the city. The most important part of solid waste management system is the collection of waste. Based on the collection system only other systems viz., transportation, processing, treatment and disposal of waste are decided. Presently, house-to-house collection system or community bin system is adopted in most of the cities in India for collection of waste. The wastes collected by these systems are heterogeneous in nature. They consist of paper, plastic, metals, glass, coconut husk, rags, clothing, vegetable, fruits, leaves and cooked food waste, crockery, wooden matter stones, bricks and so on. This creates problems and difficulties in treatment and processing of wastes. It is very difficult to segregate individual components of commingled solid waste for effective treatment and processing.

Waste can be categorized as dry waste and biodegradable wet waste. The dry waste includes paper, plastic, glass, metals, coconut husk, rags, clothing, crockery, wooden matter, stones, and bricks, etc. The biodegradable wet waste includes vegetable, fruits, green leaves, spoiled cooked food and flowers, etc.

Solid waste management may be defined as the discipline associated with the control of generation, storage, collection, transfer and transport, processing, and disposal of solid wastes in a manner that is in accord with the best principles of public health, economics, engineering, conservation aesthetics, and other environmental considerations, that is also responsive to the public attitudes.

The problems associated with the management of solid wastes are complex because of the quantity and diverse nature of the wastes, the rapid development of sprawling urban areas, the funding limitations for public services in many large cities, the impact of technologies and emerging limitations in both energy and raw materials.

The six functional elements of solid waste management system are as follows:

Waste Generation:

Waste generation encompasses activities in which materials are identified as no longer being of value and either thrown away or gathered together for disposal. Waste generation is, at present, an activity that is not very controllable.

Waste Handling and Generation, Storage and Processing at the Source:

It is the second of the six functional elements in the solid waste management system associated with the management of wastes until they are placed in storage containers for collection. Handling also encompasses movement of loaded containers to the point of collection. Separation of waste components is an important step in handling and storage of solid waste at source. For example, from the standpoint of materials specifications and revenues from the sale of recovered materials, the best place to separate waste materials for reuse and recycling is at the source of generation. House-owners are becoming more aware of the importance of separating newspapers and cardboard, bottles, yard wastes, aluminum cans, and ferrous materials. On-site storage is of primary importance because of public health concerns and aesthetic considerations. Processing at source involves activities such as compaction and yard waste composting.

Collection:

The functional element of collection includes not only gathering of solid wastes and recyclable materials, but also the transport of these materials to the location where the collection vehicle is emptied. This location may be a material processing facility, a transfer station, or a landfill disposal site. Collection system accounts for almost 50 percent of the total annual cost of urban solid waste management. This service may cost the individual homeowner Rs. 10,000/—per year or more.

Separation, Processing and Transformation of Solid Waste:

The separation, processing and transformation of solid waste materials is the fourth of the functional elements. The recovery of separated materials, the separation and processing of solid waste components, and transformation of solid waste that occur primarily in locations away from the source of waste generation are encompassed by this functional element. The types of means and facilities that are now used for the recovery of waste materials that have been separated at the source include curbside collection, drop off, and buy back centers. The separation and processing of waste that have been separated at the source and the separation of commingled wastes usually occur at materials recovery facility, transfer stations, combustion facilities and disposal sites. Processing often includes the separation of bulky items, separation of waste components by size using screens, manual separation of waste components, size reduction by shredding, separation of ferrous metals using magnets, volume reduction by compaction, and combustion.

The transformation processes are used to reduce the volume and weight of waste requiring disposal and to recover conversion products and energy. The organic fraction of municipal solid waste (MSW) can be transformed by a variety of chemical and biological processes. The most commonly used chemical transformation process is combustion, which is used in conjunction with recovery of energy in the form of heat. The most commonly used biological transformation process is aerobic composting. The selection of a given set of processes will depend on the waste management objectives to be achieved.

Transfer and Transport:

The functional element of transfer and transport involves two steps: 1) the transfer of wastes from the smaller collection vehicle to the larger transport equipment, 2) the subsequent transport of the wastes, usually over long distances, to a processing or disposal site. The transfer usually takes place in a transfer station.

Disposal:

The final functional element in the solid waste management system is disposal. In the recent times, disposal of wastes by landfilling or land spreading is the ultimate fate of all solid wastes, whether they are residential wastes collected and transported directly to a landfill site, residual materials from materials recovery facilities (MRFs), residue from the combustion of solid waste, compost or other substances from various solid waste-processing facilities. A modern sanitary landfill is not a dump; it is an engineered facility used for disposing solid wastes on land or within earth's mantle without creating nuisances or hazards to public health or safety, via, breeding of rats and insects and the contamination of groundwater.

When all the functional elements have been evaluated for use, and all of the interfaces and connections between various elements have been matched for effectiveness and economy, the community has developed an integrated waste management system. In this context integrated solid waste management (ISWM) can be defined as the selection and application of appropriate technique, and management programs to achieve specific waste management goals.

Existing Situation:

Presently, in India the conventional plastic bins without lid are in use for the storage of household waste. These bins are of two types one with aeration system and another without aeration system. There are plastic bins with mechanical opening and closing lid system. These plastic bins are closed at the bottom. In some places the waste is collected in plastic bags. The waste collected in such bin every day is disposed off in bigger community bins or it is collected by the cleaning personnel during house to house collection. The waste collected by cleaning personnel is then deposited in bigger community bins. Hence the wastes collected in such bins require disposal in community bins every or alternate day and is unavoidable. The major drawback of all the above-cited bins is that they pose unwanted foul odour. This is because the wet waste undergoes anaerobic decomposition producing gases with foul odour and leachate. The leachate produced is deposited at the bottom of the bin. This again undergoes anaerobic decomposition forming gases and organic liquids. The household bins without lid or cover pose the problem of insects and flies also. Moreover, these bins are not suitable for summer, rainy and winter seasons.

In Ahmedabad and Pune two of the major cities of India, house to house collection system is popular and adopted in entire city. The waste in these cities is segregated into dry and wet waste at source i.e. in individual houses and is collected separately during house to house collection. The cleaning personnel remove the materials, which have sale value. The remaining dry material along with wet waste is deposited in a community bin. In this way the whole concept of segregation of waste at source for its effective treatment and process is defeated.

Until now no household dust bin for the storage of biodegradable wet waste has been patented. Reference may be made to U.S. Pat. No. 7,011,224 to Sheng Bin, Hsiesh wherein soft storage bins are the containers having a body made from flexible materials such as canvas or leather. This bin has a simple collapsible structure. As the bin is made up of light material and hence easy to carry. This bin is not for the storage of waste material. However, the problem of storage waste material has been overcome in the present invention.

Reference may be made to U.S. Pat. No. 7,008,163 to Russell; Mathew wherein bulk storage bins have been designed for storing the bulk materials such as coal, ores, grain. Such storage bins can be located on a ground site or in the hold of a ship and does not address the existing problems.

Reference may be made to U.S. Pat. No. 5,403,740 to Menefee et al., wherein the biodegradable compost bin is relatively inexpensive and light in weight and are easily collapsed or folded for ease to storage and shipment. The bin is made up of a biodegradable material which is organic membrane and does not address the existing problems.

Reference may be made to U.S. Pat. No. 5,766,876 to Santiago et al., wherein compostable matter, such as food and human waste, is decomposed and effectively transformed, what is typically regarded as “waste” into useable “compost”. It is a compost bin and not a storage bin.

Numerous composting and vermicomposting devices have been patented and are disclosed for example, in U.S. Pat. No. 5,185,261 to Wassington, U.S. Pat. No. 5,413,934 to Fischer, U.S. Pat. No. 5,285,534 to Criss, U.S. Pat. No. 5,741,344 to Werkentine, U.S. Pat. No. 6,103,124 to Inoue and U.S. Pat. No. 6,576,462 to Thompson wherein the ability to process all kitchen wastes has been claimed. Unfortunately, these prior art devices possess inherent deficiencies like unsuitable size, difficult to operate and high cost. These patents do not address the existing problems.

In the prior art detailed above, the major drawbacks are the following:

Foul odor

Insects and flies

Leachate formation

Daily disposal of waste

In order to overcome the above draw backs in the existing bins, efforts were made to address them through some innovative design. The problem of foul odour, insects and flies is caused due to the formation of leachate as a result of the decomposition of waste. This can be solved by reducing the moisture and leachate formation. The best way to reduce moisture content of the decomposing waste is by providing aeration. Natural aeration system is provided by placing a mesh at the bottom of the dust bin. The problem of insects and flies is also eliminated by keeping a cover with a hole at the centre of the cover of the bin. When the bin is full after couple of days it can be very easily taken for disposal of waste. Such waste, which is rich in organic matter, if collected properly, can be processed effectively for the production of good manure. Efforts were also made so that the bin can be used in summer, rainy and winter seasons.

### OBJECTS OF THE INVENTION

The main objective of the present invention is to provide a device for the storage and treatment of biodegradable wet solid waste wherein, there is a bucket open at bottom, a mesh, a basket containing leachate absorbing media and coarse aggregate/pebble and stand which obviates the drawback of the hitherto known in the prior art as detailed above.

Another objective of the present invention is to provide methodology for segregation of household waste generated every day into dry and wet at source.

Still another objective of the present is to provide longer period for storage (detention time) of biodegradable wet waste.

Yet another objective of the present invention is to provide reduction in total cost on collection, segregation, transportation, processing, treatment and disposal of waste, if solid waste management system at urban centers is designed properly.

Another objective of the present invention is to provide less land requirement for disposal of waste, as there is land scarcity in metropolitan and big cities.

Still another objective of the present invention is to provide dust bin which occupies very small space for its easy handling and operation to individual houses.

Yet another objective of the present invention is to design dust bin very attractive so that it can be kept at any place in the house where there is proper air circulation.

Another objective of the present invention is to provide organic product, which can be used for preparing useful product that is compost.

### SUMMARY OF THE PRESENT INVENTION

Accordingly, the present invention provides a device for the storage and treatment of biodegradable wet solid waste, the said device comprising of a basket (6) with holes (7) on the lateral surface and closed at the bottom, the said basket supporting a bucket (3) with handle (4) and removable cover (2) having a hole (1) at the centre and completely open at the bottom with a circular removable mesh (5) which fits into the bucket at the bottom and the entire assembly supported on a stand (8) of circular cross section bar.

In an embodiment of the invention, the size of the hole (1) in the cover (2) of the bucket (3) is preferably in the range of 5 mm to 10 mm.

In another embodiment of the invention, the said cover (2) can be made up of any plastic or non-corrosive metal.

In an embodiment of the invention, the said bucket (3) is made up of any plastic or non-corrosive metal.

In an embodiment of the invention, the capacity of the said device is in the range of 10 liter to 25 liter.

In an embodiment of the invention, the mesh (5) size is in the range of 20 mm to 25 mm.

In an embodiment of the invention, the said mesh (5) is made up of galvanized mild steel.

In an embodiment of the invention, the height of the said basket (6) is preferably in the range of 15 cm to 20 cm.

In an embodiment of the invention, the said basket (6) is made up of any plastic or non-corrosive metal.

In an embodiment of the invention, the height of the said stand (8) is preferably in the range of 40 cm to 45 cm.

In an embodiment of the invention, the said device is provided with aeration system to overcome the problems of any foul odour, insects and flies.

In an embodiment of the invention, the wet waste stored in the said device undergoes volume reduction due to aeration and compaction.

In an embodiment of the invention, the leachate undergoes evaporation at normal temperature due to aeration.

In an embodiment of the invention, the leachate is absorbed in absorbing media which is the soil kept in the basket at the bottom through a layer of coarse aggregates or pebbles over the soil.

In an embodiment of the invention, the product obtained once the device is full can be converted to compost.

### BRIEF DESCRIPTION OF THE DRAWING

FIG. 1 shows the front view of novel design of household dust bin for the storage and treatment of biodegradable wet solid waste in accordance with the present invention with cover.

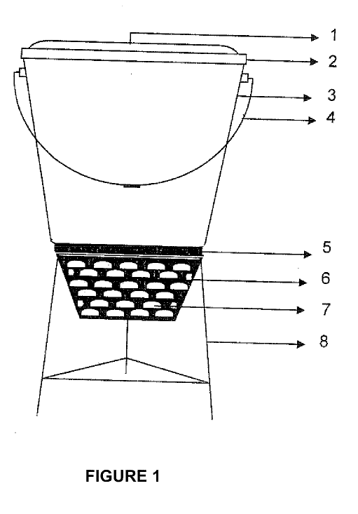

In the drawing numbers from 1 to 8 indicates corresponding parts of the drawing.

No. 1 represents the hole of size 5 mm to 10 mm at the centre of the cover and it helps in air circulation from bottom to top.

No. 2 represents the cover made of plastic or non-corrosive metal and provides protection from rats, cats, birds and flies.

No. 3 represents the bucket made of plastic or non-corrosive metal and is the main unit for storage of the wet waste. The size of the bin varies from 10 to 25 liters and is most suitable for household. The shape and the size may be different although the intention remains the same.

No. 4 represents the handle of the bucket and helps for its easy handling and movement.

No. 5 represents the mesh of size 20 mm to 25 mm and is provided at the bottom of the bucket, which is open at the bottom for resting the waste mass. The mesh is made of galvanized mild steel.

No. 6 represents the circular basket of height 15 to 20 cm made up of plastic or non-corrosive metal which is 75% filled with leachate absorbing media. Soil is used as an absorbing media. Above the soil layer the remaining portion is filled with pebbles or coarse aggregates of size about 20 mm to 35 mm for aeration. The leachate generated due to decomposition passes through pebble or coarse aggregate layer and then absorbed by the soil. The basket is made perforated by holes along the lateral surface.

No. 7 represents the holes in the basket for aeration. The holes can be of any size. It may be i) circular with 16 mm to 20 mm diameter ii) semi circular with 20 mm to 25 mm diameter and height 5 to 8 mm or iii) rectangular of size 20 mm to 25 mm in length and 5 mm to 8 mm in breadth. The leachate absorbed in the soil gets dried due to aeration.

No. 8 represents the M.S. stand, which provides convenient height of 40 to 45 cm. to the user for easy operation.

### DETAILED DESCRIPTION OF THE INVENTION

In order to have sustainable solid waste management system, one must have effective treatment and disposal methodologies. Owing to heterogeneous nature of the wastes, these methodologies are not effective. In order to make them effective, the waste has to be separated into a) dry waste and b) biodegradable wet waste at the source of generation i.e. at household level. The storage of dry waste is not a problem. By conducting several experiments, it is observed that the dry waste can be collected separately and stored up to 6 months. This waste occupies very small space in the house. The quantity of dry waste generated during the period of 6 months ranges between 15 to 20 kg. The dry waste generated, if collected periodically like once in a month to six month can be segregated into combustibles and non-combustibles. The treatment processes like, incineration; pyrolysis or gasification can be used for treating this waste effectively. With such effective treatment processes, one can generate energy from waste.

The most difficult part of such segregation is wet waste. One cannot keep the wet waste more than 24 hours in a house. However, if air is circulated through a heap of wet waste, it reduces the odor problem. The waste on its own weight goes on settling. This causes reduction in volume. If the wet waste mass is huge, then a foul odor may be generated and the volume reduction takes longer time. Leachate is also generated during the process. If this system is decentralized and the wet waste is stored at individual houses, then the odor and volume reduction can be achieved at a much faster rate. Keeping this novel concept in mind, this household dust bin was designed. This system keeps the wet waste aerated, allows the wet waste to settle and reduces the odor and leachate formed. In case some leachate is generated, then it is absorbed in the absorbing media, which is also provided with the aeration system.

To operate the device, the consumer adds household wet waste to the bucket by removing the cover from the top and replacing the cover. Micro organisms in air deposited and already present in the waste initiate the decomposition of waste in the presence of air. The average waste generation rate is taken 0.240 kg/family/day. The size of the family is considered to be 5 persons. With this rate of generation a family can store the wet waste for a period of one month. The quantity of waste to be stored in a month will be about 8.0 kg. The average density of waste is taken as 400 kg/m³. As one go on putting the waste in the bucket the density of waste goes on increasing due to self compaction. The bin is flexible to accommodate the waste, if the rate of waste generation exceeds marginally during special occasions.

The developed novel design of household dust bin for the storage and treatment of biodegradable wet-solid waste works on the aeration system. As the size of the mesh kept at the bottom of the bucket is large, i.e. 20 mm to 25 mm, initially the leafy vegetable waste or thin layer of the grass may be put on the mesh so that the waste having particle size less than 20 mm to 25 mm will not fall down. Then the wet waste generated can be loaded with lid closed daily. It is preferable to keep the bin open during day time and closed during night. When the bin is full then the waste can be disposed and again used as above. Depending upon the frequency of collection, i.e. once in a week, twice a month or once in a month, the waste can be disposed-off even before filling of the dust bin.

### SIGNIFICANCE OF PRESENT INVENTION

The most important utility of this invention is that it helps in segregation of biodegradable wet waste and dry waste at source (household level). Segregation of waste at source will help in effective treatment and processing of both dry as well as biodegradable wet waste. Advantage of this invented bin is that the biodegradable wet waste can be stored and treated for a longer period as it undergoes volume reduction due to self compaction and through moisture loss due to aeration without giving any foul odor and other problems like insects, flies, leachate, etc. The leachate generated by decomposition and compaction gets reduced due to aeration in room temperature. The storage of waste for longer period reduces the time spent on daily disposal of household waste. Another advantage of this bin is that it gives the product, which can be processed further by existing composting methodology converting the waste into good compost. Similarly the segregated dry waste can also be stored in big plastic bags for longer period. Hence, dry waste and wet waste can be collected once in a month separately.

First take the bucket with a cover. The size of this bucket will depend upon the detention time of waste i.e. how long the wet waste is allowed to keep in the bin. Remove the bottom of the bucket by cutting. Make a small hole at the centre of the cover.

Take a welded mesh and cut it according to the shape of the container so that, it fits into the bottom of the container. A bucket is used in this case. This mesh is removable at the time of cleaning the bucket.

Take a basket 15 to 25 cms height, which will fit to the bottom of the container. The basket should have perforation on the lateral surface. If it is not perforated, then make it perforated by making holes on lateral surface. These holes are required for keeping the system aerated. The basket is closed at the bottom.

The basket is filled 75% with soil at the bottom to absorb the leachate generated. The remaining portion of the basket is filled with pebbles or coarse aggregates of appropriate sizes, preferably about 25 to 30 mm.

Take a circular tripod stand. The shape and size of the top should be such that the basket should fit and rest on it. The stand should have an arrangement to support the basket from the bottom. The height of the stand ranges from 45 to 50 cm.

The present invention is illustrated in the Figure no. 1, although it is not restricted to such a shape only.

The following examples are given by way of illustration of working of the invention in actual practice and therefore should not be constructed to limit the scope of the present invention.

### EXAMPLE 1

A wet waste generated in a house generally consists of vegetable waste, fruit waste, left over cooked food, used or waste flowers, green leaves, spoiled food, vegetables and eatables. Such materials have been stored in the dust bin.

The wet waste stored in the bin has been tested for organoliftic test for bad odour. This test was conducted by a group of panelist and it was observed that the stored waste does not pose any foul odour; even it is stored for 7-30 days.

The experiment of storage of wet waste was conducted in all the three seasons viz., summer, rainy and winter seasons. It was observed that the bin works satisfactorily in all the seasons.

Experiments were also conducted for minimum and maximum period for the storage of wet waste. It was observed that the bin takes minimum 7 days and maximum 45 days for filling. The period mostly depends on the size of the bin and the quality and quantity of wet waste generated per day.

Experiments were conducted by using 11 lit. and 20 lit. dust bin. It was observed that the bin works very nicely in both. These sizes are most suitable for small families/flats for the storage and treatment of wet waste.

As the function of the bin is based on aerobic system and requires airflow, the bin should be placed in an open space, where there is a flow of air.

As the bin occupies very small space and waste can be stored up to 30 days. It is very useful for those who are staying in multistoried buildings.

The wet waste stored in the bin is rich in organic matter and after treatment provides good manure/compost with further treatment. The compost prepared contains 2-3% nitrogen and C/N ratio ranges from 15 to 18.

No expenditure is incurred on operation and maintenance of the bin as nothing is added to the bin when it is in use.

### EXAMPLE 2

The bin was also used for storing the flower waste and it works nicely as above. To summarize, the novel design of the household dust bin for storage of biodegradable wet waste is very attractive and occupies very small area/space in the house. The total height of the bin is 78 to 80 cms and hence provides easy operation of the bin. One need not have to bend for opening the bin, putting waste in the bin and closing the bin. As the bin is capable of storing the waste for a longer period, a person need not have to go out for disposal of the waste every day. This saves the daily time spent on the disposal of waste. If the use of such bins is implemented in an area, then it will reduce the total cost incurred on collection, transportation and disposal. Such waste, which is rich in organic matter, if collected properly, can be processed effectively for the production of good manure. The novelty of the bin is that the wet waste can be stored for a longer period and it does not caused any foul odour problem. The bin is also capable of arresting the leachate problem.

### ADVANTAGES

The main advantages of the present invention are

1. The wet waste can be stored for a longer period (from 1 week to 6 weeks)

2. The wet waste stored does not pose the problems of foul odour, insects and flies

3. The leachate generated if any is absorbed in absorbing media

4. It reduces the cost incurred on solid waste management system

5. It provides very good raw material for treatment process like compost

6. It helps in providing less area for treatment processes

7. It also helps in providing less area for disposal

8. It is very handy and easy for operation

9. There is no operation and maintenance cost

1. A device for the storage and treatment of biodegradable wet solid waste, the said device comprising of a basket (6) with holes (7) on the lateral surface and closed at the bottom, the said basket supporting a bucket (3) with handle (4) and removable cover (2) having a hole (1) at the centre and completely open at the bottom with a circular removable mesh (5), which fits into the bucket at the bottom and the entire assembly supported on a stand (8) of circular cross section bar.

2. A device for the storage and treatment of biodegradable wet solid waste according to claim 1 wherein, the size of the hole (1) in the cover (2) of the bucket (3) is preferably in the range of 5 mm to 10 mm.

3. A device for the storage and treatment of biodegradable wet solid waste according to claim 1 wherein, the said cover (2) can be made up of any plastic or non-corrosive metal.

4. A device for the storage and treatment of biodegradable wet solid waste according to claim 1 wherein, the said bucket (3) is made up of any plastic or non-corrosive metal.

5. A device for the storage and treatment of biodegradable wet solid waste according to claim 1 wherein, the capacity of the said device is in the range of 10 liter to 25 liter.

6. A device for the storage and treatment of biodegradable wet solid waste according to claim 1 wherein, the mesh (5) size is in the range of 20 mm to 25 mm.

7. A device for the storage and treatment of biodegradable wet solid waste according to claim 1 wherein, the said mesh (5) is made up of galvanized mild steel.

8. A device for the storage and treatment of biodegradable wet solid waste according to claim 1 wherein, the height of the said basket (6) is preferably in the range of 15 cm to 20 cm.

9. A device for the storage and treatment of biodegradable wet solid waste according to claim 1 wherein, the said basket (6) is made up of any plastic or non-corrosive metal.

10. A device for the storage and treatment of biodegradable wet solid waste according to claim 1 wherein, the height of the said stand (8) is preferably in the range of 40 cm to 45 cm.

11. A device for the storage and treatment of biodegradable wet solid waste according to claim 1 wherein, the said device is provided with aeration system to overcome the problems of any foul odour, insects and flies.

12. A device for the storage and treatment of biodegradable wet solid waste according to claim 1 wherein, the wet waste stored in the said device undergoes volume reduction due to aeration and compaction.

13. A device for the storage and treatment of biodegradable wet solid waste according to claim 1 wherein, the leachate undergoes evaporation at normal temperature due to aeration.

14. A device for the storage and treatment of biodegradable wet solid waste according to claim 1 wherein, the leachate is absorbed in absorbing media which is the soil kept in the basket at the bottom through a layer of coarse aggregates or pebbles over the soil.

15. A device for the storage and treatment of biodegradable wet solid waste according to claim 1 wherein, the product obtained once the device is full can be converted to compost.
